(19) United States
(12) Patent Application Publication
(10) Pub. No.: US 2009/0005606 A1
(43) Pub. Date: Jan. 1, 2009

(54) HIGH SHEAR PROCESS FOR THE PRODUCTION OF CUMENE HYDROPEROXIDE

(75) Inventors: Abbas Hassan, Sugar land, TX (US); Ebrahim Bagherzadeh, Sugar land, TX (US); Rayford G. Anthony, College Station, TX (US); Gregory Borsinger, Chatham, NJ (US); Aziz Hassan, Sugar Land, TX (US)

Correspondence Address: CONLEY ROSE, P.C.;David A. Rose, P. O. BOX 3267, HOUSTON, TX, 77253-3267, US

(73) Assignee: H R D CORPORATION, Houston, TX (US)

(21) Appl. No.: 12/137,465

(22) Filed: Jun. 11, 2008

Related U.S. Application Data
(60) Provisional application No. 60/946,529, filed on Jun. 27, 2007.

(51) Int. Cl. C07C 407/00; B01J 19/00

(52) U.S. Cl. 568/569; 422/187

(57) ABSTRACT

Use of a high shear mechanical device incorporated into a process for the production of cumene hydroperoxide as a mixer/reactor device is capable of decreasing mass transfer limitations, thereby enhancing the cumene hydroperoxide production process. A system for the production of cumene hydroperoxide from oxidation of cumene, the system comprising a reactor and an high shear mixer the outlet of which is fluidly connected to the inlet of the reactor; the high shear mixer capable of providing a dispersion air gas bubbles within a liquid, the bubbles having an average bubble diameter of less than about 100 μm.

### CROSS-REFERENCE TO RELATED APPLICATIONS

This application claims the benefit under 35 U.S.C. § 119(e) of U.S. Provisional Patent Application No. 60/946,529 filed Jun. 27, 2007, the disclosure of which is hereby incorporated herein by reference.

### STATEMENT REGARDING FEDERALLY SPONSORED RESEARCH OR DEVELOPMENT

Not Applicable.

### BACKGROUND OF THE INVENTION

1. Technical Field

The present disclosure generally relates to the production of cumene hydroperoxide by oxidation of cumene, and more particularly to apparatus and methods for producing cumene hydroperoxide via air oxidation of cumene. More specifically the disclosure relates to the reduction of mass transfer limitations in apparatus and methods of oxidizing cumene to form cumene hydroperoxide.

2. Background of the Invention

The cumene process involves the production of industrial significant products acetone and phenol from benzene and propylene. Reactants required for the cumene process include gaseous oxygen and small amounts of an initiator, cumene hydroperoxide. Cumene hydroperoxide (hereinafter, CHP) is a precursor for phenol production in the cumene process.

Cumene is formed in the gas phase Friedel-Crafts alkylation of benzene with propylene. Cumene is used form cumene hydroperoxide by a liquid phase oxidation reaction. The decomposition of cumene hydroperoxide produces a mole of acetone per mole of phenol. CHP has other commercial uses, such as an initiator of radicals, which create cumene hydroperoxide with high selectivity. In these applications, high selectivity minimizes the formation of byproducts that would hinder its use as a radical initiator.

Free radical cumene oxidation reactions are conventionally conducted in the presence of a water phase by the “heterogeneous wet oxidation” method. Alternatively, the radical cumene oxidation is conducted in anhydrous conditions by the “dry oxidation” method. U.S. Patent Application 2006/0014985 describes an anhydrous process for the synthesis of cumene hydroperoxide by oxidation of cumene with oxygen, in the presence of a basic medium insoluble in the reaction environment, for example a pyridinic resin. The presence of water improves safety and control of the exothermic reaction, and may reduce capital investment.

Conventionally, the heterogeneous wet oxidation method in commercial applications is a continuous process using a cascade of at least two gas-sparged reactors with a variable temperature profile. The main oxidation reaction products are CHP, dimethylbenzyl alcohol and acetophenone. Trace amounts of acidic byproducts, such as formic acid, acetic acid, and phenol, inhibit the oxidation reaction resulting in a decrease in both rate, yield and negatively affecting CHP selectivity. U.S. Pat. Nos. 3,187,055; 3,523,977; 3,687,055; 6,043,399; and 3,907,901 teach that alkali metal bases, such as sodium hydroxide (NaOH), and bicarbonate salts of alkali metals, such as sodium carbonate (Na₂CO₃), can be used as additives to remove the trace acid impurities.

A process for the preparation of cumene hydroperoxide is described in U.S. Pat. No. 6,043,399 which discloses liquid phase oxidation of cumene in the presence of at least one agent chosen from the hydroxide or carbonate of an alkali metal and/or an alkaline-earth metal.

Accordingly, there is a need in the industry for improved process of cumene hydroperoxide production, whereby production rates are increased, reaction rates are improved, and reaction conditions such as lower temperature and pressure, are commercially feasible.

### SUMMARY OF THE INVENTION

A high shear system and process for enhancing the production of cumene hydroperoxide is disclosed. The high shear process reduces mass transfer limitations, thereby increasing the effective reaction rate and allowing reactor operation at reduced temperature and pressure, with a reduction in contact time and/or an increase in product yield. In accordance with certain embodiments of the present disclosure, a process is provided that makes possible an increase in the rate of liquid phase production of cumene hydroperoxide by providing for more optimal time, temperature and pressure conditions than are conventionally used.

In an embodiment described in the present disclosure, a process employs a high shear device to provide enhanced time, temperature and pressure reaction conditions resulting in accelerated chemical reactions between multiphase reactants. Further, a process disclosed in an embodiment described herein, comprises the use of a high shear device to provide for the production of CHP without the need for heterogeneous wet oxidation reactors.

These and other embodiments, features, and advantages will be apparent in the following detailed description and drawings.

### BRIEF DESCRIPTION OF THE DRAWINGS

For a more detailed description of the preferred embodiment of the present invention, reference will now be made to the accompanying drawings, wherein:

FIG. 1 is a cross-sectional diagram of a high shear device for the production of cumene hydroperoxide.

FIG. 2 is a process flow diagram according to an embodiment of the present disclosure comprising a high shear process for the production of cumene hydroperoxide.

### DETAILED DESCRIPTION OF THE PREFERRED EMBODIMENTS

### Overview

An improved process and system for the production of cumene hydroperoxide employs an external or in-line high shear device. The high shear device is a mechanical reactor, mixer, or mill to provide rapid contact and mixing of chemical reactants in a controlled environment in the device. The high shear device reduces the mass transfer limitations on the reaction and thus increases the overall reaction rate.

Chemical reactions involving liquids, gases, solids, and catalysts rely on the laws of kinetics that involve time, temperature, and pressure to define the rate of reactions. In cases where it is desirable to react two or more raw materials of different phases, for example, a solid and liquid; liquid and gas; solid, liquid, and gas, one of the limiting factors in controlling the rate of reaction involves the contact time of the reactants. In the case of heterogeneously catalyzed reactions there is the additional rate limiting factor of having the reacted products removed from the surface of the catalyst to enable the catalyst to catalyze further reactants.

In conventional reactors, the contact time for the reactants and/or catalyst is often controlled by mixing, which provides contact between two or more reactants involved in a chemical reaction. A reactor assembly that comprises a high shear device reduces mass transfer limitations and thereby allows the reaction to more closely approach the intrinsic kinetic rate. When effective reaction rates are accelerated, residence times may be decreased, thereby increasing the throughput obtainable by the system. Alternatively, where the present yield is acceptable, decreasing the required residence time allows for the use of less severe temperatures and/or pressures than conventional processes. Alternatively or additionally, yield of product may be increased via the high shear system and process.

### High Shear Device

High shear devices (HSD) such as a high shear mixer, or high shear mill, are generally divided into classes based upon their ability to mix fluids. Mixing is the process of reducing the size of inhomogeneous species or particles within the fluid. One metric for the degree or thoroughness of mixing is the energy density per unit volume that the mixing device generates to disrupt the fluid particles. The classes are distinguished based on delivered energy density. There are three classes of industrial mixers having sufficient energy density to consistently produce mixtures or emulsions with particle, globule, or bubble sizes in the range of 0 to 50 μm

Homogenization valve systems are typically classified as high energy devices. Fluid to be processed is pumped under very high pressure through a narrow-gap valve into a lower pressure environment. The pressure gradients across the valve and the resulting turbulence and cavitations act to break-up any particles in the fluid. These valve systems are most commonly used in milk homogenization and can yield average particle size range from about 0.01 μm to about 1 μm. At the other end of the spectrum are high shear mixer systems classified as low energy devices. These systems usually have paddles or fluid rotors that turn at high speed in a reservoir of fluid to be processed, which in many of the more common applications is a food product. These systems are usually used when average particle, or bubble, sizes of greater than 20 microns are acceptable in the processed fluid.

Between low energy-high shear mixers and homogenization valve systems, in terms of the mixing energy density delivered to the fluid, are colloid mills, which are classified as intermediate energy devices. The typical colloid mill configuration includes a conical or disk rotor that is separated from a complementary, liquid-cooled stator by a closely-controlled rotor-stator gap, which is maybe between 0.025 mm and 10.0 mm. Rotors are usually driven by an electric motor through a direct drive or belt mechanism. Many colloid mills, with proper adjustment, can achieve average particle, or bubble, sizes of about 0.01 μm to about 25 μm in the processed fluid. These capabilities render colloid mills appropriate for a variety of applications including colloid and oil/water-based emulsion processing such as that required for cosmetics, mayonnaise, silicone/silver amalgam formation, or roofing-tar mixing.

An approximation of energy input into the fluid (kW/L/min) can be made by measuring the motor energy (kW) and fluid output (L/min). In embodiments, the energy expenditure of a high shear device is greater than 1000 W/m³. In embodiments, the energy expenditure is in the range of from about 3000 W/m³ to about 7500 W/m³. The shear rate generated in a high shear device may be greater than 20,000 s⁻¹. In embodiments, the shear rate generated is in the range of from 20,000 s⁻¹ to 100,000 s⁻¹.

Tip speed is the velocity (m/sec) associated with the end of one or more revolving elements that is transmitting energy to the reactants. Tip speed, for a rotating element, is the circumferential distance traveled by the tip of the rotor per unit of time, and is generally defined by the equation V (m/sec)=π·D·n, where V is the tip speed, D is the diameter of the rotor, in meters, and n is the rotational speed of the rotor, in revolutions per second. Tip speed is thus a function of the rotor diameter and the rotation rate. Also, tip speed may be calculated by multiplying the circumferential distance transcribed by the rotor tip, 2πR, where R is the radius of the rotor (meters, for example) times the frequency of revolution (for example revolutions per minute, rpm).

For colloid mills, typical tip speeds are in excess of 23 m/sec (4500 ft/min) and can exceed 40 m/sec (7900 ft/min). For the purpose of the present disclosure the term ‘high shear’ refers to mechanical rotor-stator devices, such as mills or mixers, that are capable of tip speeds in excess of 5 m/sec (1000 ft/min) and require an external mechanically driven power device to drive energy into the stream of products to be reacted. A high shear device combines high tip speeds with a very small shear gap to produce significant friction on the material being processed. Accordingly, a local pressure in the range of about 1000 MPa (about 145,000 psi) to about 1050 MPa (152,300 psi) and elevated temperatures at the tip of the shear mixer are produced during operation. In certain embodiments, the local pressure is at least 1034 MPa. The local pressure further depends on the tip speed, fluid viscosity, and the rotor-stator gap during operation.

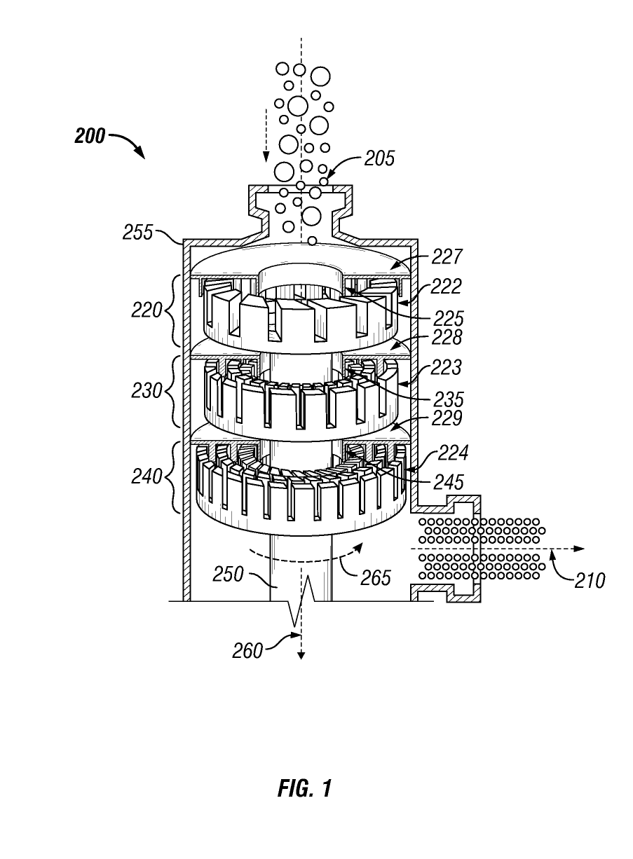

Referring now to FIG. 1, there is presented a schematic diagram of a high shear device 200. High shear device 200 comprises at least one rotor-stator combination. The rotor-stator combinations may also be known as generators 220, 230, 240 or stages without limitation. The high shear device 200 comprises at least two generators, and most preferably, the high shear device comprises at least three generators.

The first generator 220 comprises rotor 222 and stator 227. The second generator 230 comprises rotor 223, and stator 228; the third generator comprises rotor 224 and stator 229. For each generator 220, 230, 240 the rotor is rotatably driven by input 250. The generators 220, 230, 240 rotate about axis 260 in rotational direction 265. Stator 227 is fixably coupled to the high shear device wall 255.

The generators include gaps between the rotor and the stator. The first generator 220 comprises a first gap 225; the second generator 230 comprises a second gap 235; and the third generator 240 comprises a third gap 245. The gaps 225, 235, 245 are between about 0.025 mm (0.01 in) and 10.0 mm (0.4 in) wide. Alternatively, the process comprises utilization of a high shear device 200 wherein the gaps 225, 235, 245 are between about 0.5 mm (0.02 in) and about 2.5 mm (0.1 in). In certain instances the gap is maintained at about 1.5 mm (0.06 in). Alternatively, the gaps 225, 235, 245 are different between generators 220, 230, 240. In certain instances, the gap 225 for the first generator 220 is greater than about the gap 235 for the second generator 230, which is greater than about the gap 245 for the third generator 240.

Additionally, the width of the gaps 225, 235, 245 may comprise a coarse, medium, fine, and super-fine characterization. Rotors 222, 223, and 224 and stators 227, 228, and 229 may be toothed designs. Each generator may comprise two or more sets of rotor-stator teeth, as known in the art. Rotors 222, 223, and 224 may comprise a number of rotor teeth circumferentially spaced about the circumference of each rotor. Stators 227, 228, and 229 may comprise a number of stator teeth circumferentially spaced about the circumference of each stator. In embodiments, the inner diameter of the rotor is about 11.8 cm. In embodiments, the outer diameter of the stator is about 15.4 cm. In further embodiments, the rotor and stator may have an outer diameter of about 60 mm for the rotor, and about 64 mm for the stator. Alternatively, the rotor and stator may have alternate diameters in order to alter the tip speed and shear pressures. In certain embodiments, each of three stages is operated with a super-fine generator, comprising a gap of between about 0.025 mm and about 3 mm. When a feed stream 205 including solid particles is to be sent through high shear device 200, the appropriate gap width is first selected for an appropriate reduction in particle size and increase in particle surface area. In embodiments, this is beneficial for increasing catalyst surface area by shearing and dispersing the particles.

High shear device 200 is fed a reaction mixture comprising the feed stream 205. Feed stream 205 comprises an emulsion of the dispersible phase and the continuous phase. Emulsion refers to a liquefied mixture that contains two distinguishable substances (or phases) that will not readily mix and dissolve together. Most emulsions have a continuous phase (or matrix), which holds therein discontinuous droplets, bubbles, and/or particles of the other phase or substance. Emulsions may be highly viscous, such as slurries or pastes, or may be foams, with tiny gas bubbles suspended in a liquid. As used herein, the term “emulsion” encompasses continuous phases comprising gas bubbles, continuous phases comprising particles (e.g., solid catalyst), continuous phases comprising droplets of a fluid that is substantially insoluble in the continuous phase, and combinations thereof.

Feed stream 205 may include a particulate solid catalyst component. Feed stream 205 is pumped through the generators 220, 230, 240, such that product dispersion 210 is formed. In each generator, the rotors 222, 223, 224 rotate at high speed relative to the fixed stators 227, 228, 229. The rotation of the rotors pumps fluid, such as the feed stream 205, between the outer surface of the rotor 222 and the inner surface of the stator 227 creating a localized high shear condition. The gaps 225, 235, 245 generate high shear forces that process the feed stream 205. The high shear forces between the rotor and stator functions to process the feed stream 205 to create the product dispersion 210. Each generator 220, 230, 240 of the high shear device 200 has interchangeable rotor-stator combinations for producing a narrow distribution of the desired bubble size, if feedstream 205 comprises a gas, or globule size, if feedstream 205 comprises a liquid, in the product dispersion 210.

The product dispersion 210 of gas particles, or bubbles, in a liquid comprises an emulsion. In embodiments, the product dispersion 210 may comprise a dispersion of a previously immiscible or insoluble gas, liquid or solid into the continuous phase. The product dispersion 210 has an average gas particle, or bubble, size less than about 1.5 μm; preferably the bubbles are sub-micron in diameter. In certain instances, the average bubble size is in the range from about 1.0 μm to about 0.1 μm. Alternatively, the average bubble size is less than about 400 nm (0.4 μm) and most preferably less than about 100 nm (0.1 μm).

The high shear device 200 produces a gas emulsion capable of remaining dispersed at atmospheric pressure for at least about 15 minutes. For the purpose of this disclosure, an emulsion of gas particles, or bubbles, in the dispersed phase in product dispersion 210 that are less than 1.5 μm in diameter may comprise a micro-foam.

Not to be limited by a specific theory, it is known in emulsion chemistry that sub-micron particles, or bubbles, dispersed in a liquid undergo movement primarily through Brownian motion effects. The bubbles in the emulsion of product dispersion 210 created by the high shear device 200 may have greater mobility through boundary layers of solid catalyst particles, thereby facilitating and accelerating the catalytic reaction through enhanced transport of reactants.

The rotor is set to rotate at a speed commensurate with the diameter of the rotor and the desired tip speed as described above. Transport resistance is reduced by incorporation of high shear device 200 such that the velocity of the reaction is increased by at least about 5%. Alternatively, the high shear device 200 comprises a high shear colloid mill that serves as an accelerated rate reactor (ARR). The accelerated rate reactor comprises a single stage dispersing chamber. The accelerated rate reactor comprises a multiple stage inline disperser comprising at least 2 stages.

Selection of the high shear device 200 is dependent on throughput requirements and desired particle or bubble size in the outlet dispersion 210. In certain instances, high shear device 200 comprises a Dispax Reactor® of IKA® Works, Inc. Wilmington, N.C. and APV North America, Inc. Wilmington, Mass. Model DR 2000/4, for example, comprises a belt drive, 4M generator, PTFE sealing ring, inlet flange 1″ sanitary clamp, outlet flange ¾″ sanitary clamp, 2 HP power, output speed of 7900 rpm, flow capacity (water) approximately 300-700 l/h (depending on generator), a tip speed of from 9.4-41 m/s (about 1850 ft/min to about 8070 ft/min). Several alternative models are available having various inlet/outlet connections, horsepower, nominal tip speeds, output rpm, and nominal flow rate.

Without wishing to be limited to a particular theory, it is believed that the level or degree of high shear mixing is sufficient to increase rates of mass transfer and may also produce localized non-ideal conditions that enable reactions to occur that would not otherwise be expected to occur based on Gibbs free energy predictions. Localized non ideal conditions are believed to occur within the high shear device resulting in increased temperatures and pressures with the most significant increase believed to be in localized pressures. The increase in pressures and temperatures within the high shear device are instantaneous and localized and quickly revert back to bulk or average system conditions once exiting the high shear device. In some cases, the high shear mixing device induces cavitation of sufficient intensity to dissociate one or more of the reactants into free radicals, which may intensify a chemical reaction or allow a reaction to take place at less stringent conditions than might otherwise be required. Cavitation may also increase rates of transport processes by producing local turbulence and liquid micro-circulation (acoustic streaming). An overview of the application of cavitation phenomenon in chemical/physical processing applications is provided by Gogate et al., “Cavitation: A technology on the horizon,” Current Science 91 (No. 1): 35-46 (2006). The high shear mixing device of certain embodiments of the present system and methods is operated under what is believed to be cavitation conditions effective to dissociate the cumene into free radicals exposed to oxygen for the formation of the cumene hydroperoxide product.

### Process and System for High Shear Production of Cumene Hydroperoxide

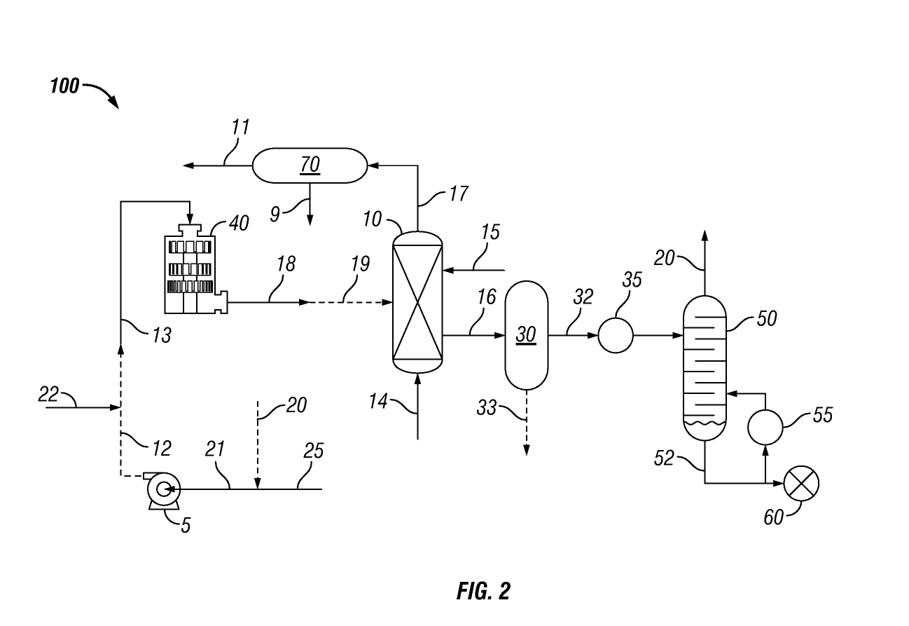

The high shear cumene hydroperoxide production process and system of the present disclosure will now be described in relation to process flow diagram illustrated in FIG. 2. FIG. 2 illustrates the basic components of a representative high shear system (HSS) 100 for producing cumene hydroperoxide (CHP). These components comprise high shear device (HSD) 40, reactor 10, and pump 5. The use of dotted lines in FIG. 2 is used to point out that additional steps that may be incorporated between reactor 10, high shear device 40, and pump 5. In certain embodiments, the dotted steps are optional.

HSS 100 may comprise more than one high shear device 40 and more than one reactor 10. For example, HSS comprises at least one high shear device 40 upstream of each reactor 10. The cumene may be oxidized in a plurality of reactors 10. Reactors 10 may be arranged in parallel, or in series. In certain configurations HSS 100 comprises from about two to about eight reactors 10.

Pump 5 is used to provide a controlled flow throughout high shear system 100. Pump 5 builds pressure and feeds high shear device 40. Pump 5 increases the pressure of the pump inlet liquid stream 21 to greater than about 203 kPa, and alternately, the pressure is greater than about 2025 kPa. Pump inlet stream 21 comprises fresh cumene 25 and recycled cumene 20, 9, as described hereinbelow. In embodiments, fresh cumene 25 is produced from the reaction of benzene and propylene, as known to those of skill in the art. A description of a suitable process for the production of fresh cumene stream 25 is found, for example, in U.S. Patent Application No. 2006/0281958, hereby incorporated by reference for all purposes.

The pressurized stream 12 exits pump 5. The increased pressure may be used to accelerate reactions. The limiting factor for pressurized stream 12 may be the pressure limitations of pump 5 and high shear device 40. Preferably, all contact parts of pump 5 comprise stainless steel. Pump 5 may be any suitable pump, for example, a Roper Type 1 gear pump, Roper Pump Company (Commerce Ga.) or a Dayton Pressure Booster Pump Model 2P372E, Dayton Electric Co (Niles, Ill.). Pressurized stream 12 is fed to high shear device inlet stream 13.

Dispersible gas stream 22 is injected into pressurized stream 12 for the production of CHP. The oxidation of cumene is carried out in the presence of a gas containing oxygen. For this purpose, it is possible to use any pure or dilute oxygen source, such as air, optionally enriched in oxygen. In embodiments, dispersible gas stream 22 comprises air. Alternatively, dispersible gas stream 22 comprises oxygen. In certain instances, dispersible gas stream 22 comprises oxygen-enriched air. Dispersible gas stream 22 and pressurized stream 12 are introduced separately or mixed to form the inlet feed stream 13 of high shear device 40. Dispersible gas stream 22 may be fed continuously into pressurized stream 12 to form inlet feed stream 13.

As discussed in detail above, the high shear device (HSD) 40 is a mechanical device that utilizes, for example, a rotor-stator mixing head with a gap between the stator and rotor. In embodiments there may be several high shear devices 40 used in series. In HSD 40, dispersible gas stream 22 and pressurized stream 12 are highly dispersed to form an emulsion comprising an average gas particle, or bubble, diameter less than about 1.5 μm; preferably the bubble diameters are about sub-micron. In certain instances, the average bubble diameter is in the range from about 1.0 μm to about 0.1 μm. Alternatively, the average bubble diameter is less than about 400 nm (0.4 μm) and most preferably less than about 100 nm (0.1 μm).

In certain instances, the high shear device 40 is incorporated into an established process, thereby enabling an increase in production (i.e., greater throughput). Without wishing to be limited to a particular theory, it is believed that the level or degree of high shear mixing is sufficient to increase rates of mass transfer and also produces localized non-ideal conditions that enable the reactions to occur that would not otherwise be expected to occur based on Gibbs free energy predictions. The localized non ideal conditions are believed to occur within the high shear device resulting in increased temperatures and pressures with the most significant increase believed to be in localized pressures. The increase in pressures and temperatures within the high shear device are instantaneous and localized and quickly revert back to average system conditions once exiting the high shear device.

The emulsion exits HSD 40 by outlet stream 18. Outlet stream 18 is introduced into reactor inlet stream 19. The reactor inlet stream 19 may be heated or cooled to maintain effective reaction temperature. Reactor inlet stream 19 enters reactor 10 for CHP production. In embodiments, CHP production is continuous in reactor 10. Reactor 10 may be any type of reactor configured for the oxidation of cumene as known to one skilled in the art, for example a fixed bed reactor. In embodiments, cumene oxidation is performed anhydrously, and reactor 10 comprises an insoluble basic medium, for example, a pyridinic resin.

Reactor 10 may be configured for maintaining higher than about atmospheric temperature. In certain instances the reactor pressure may be between about 100 kPa and about 300 kPa. Also, the reactor 10 is configured to maintain a reaction temperature that is between about 70° C. and about 120° C. In some embodiments, the temperature is between about 75° C. and about 90° C. It should be noted that the reaction temperature may vary within the reactor 10 and in certain instances the temperature decreases when the concentration of cumene hydroperoxide increases. Alternative means to maintain the reaction temperature in the reactor 10 may include a thermal jacket or coil disposed around reactor 10.

To maintain favorable reaction temperatures, HSS 100 may comprise heat exchangers. Suitable heat exchangers include plate, coil, and shell and tube designs, without limitation. Suitable locations for heat exchangers include, but are not limited to, between the reactor 10 and the pump 5; between the pump 5 and the HSD 40; between HSD 40 and the reactor 10.

In certain instances, HSS 100 comprises second inlet stream 15, comprising an aqueous solution. Second inlet stream 15 may be injected or fed directly into reactor 10. In further instances, second inlet stream 15 may be injected into HSS 100 in alternative locations. Second inlet stream 15 comprises a neutralizing agent chosen from a group consisting of hydroxides or carbonates of alkali and/or alkaline-earth metals. Preferably, the neutralizing agent is selected from alkali metals, such as sodium hydroxide, potassium hydroxide, sodium carbonate and potassium carbonate, without limitation. The quantity of neutralizing agent in second inlet stream 15 is between about 1 ppb and about 20 ppb, preferably between about 2 ppb and about 10 ppb. For example, when the neutralizing agent comprises sodium hydroxide, it does not exceed about 10 ppb, with respect to the amount of cumene introduced. In embodiments, a pH agent is injected such that the pH of the reaction mixture remains between about pH 2 and about pH 7, preferably between about pH 3 and about pH 5.

A neutralizing agent as described, for example, in U.S. Pat. No. 6,043,399, hereby incorporated herein by reference in its entirety for all purposes, may be added via second inlet stream 15. Alternatively, second inlet stream 15 may comprise ammonia, as disclosed, for example, in U.S. Pat. No. 6,620,974, incorporated herein in its entirety.

Reactor 10 further comprises gas inlet 14 for introducing gas containing oxygen. The oxygen gas thereby enhancing mixing of immiscible phases. Generally, to optimize the phase mixing, gas inlet is disposed at or near bottom of the reactor 10. Reactor 10, further comprising a gas exit 17 is configured for the removal of gas from the reactor 10. The vented gases from the reactor via gas exit 17 are kept at below about 10% oxygen, preferably between 2% and 6.5% oxygen, and most preferably between 4.5% and 6.5% oxygen. Gas exit 17 is connected to reactor 10 for removal of gas containing unreacted oxygen, any other reaction gases and/or pressure. Gas exit 17 may vent the head space of the reactor 10. Gas exit 17 may comprise a compressor, or other device as known to one skilled in the art, to compress gasses removed from the reactor 10. Additionally, gas exit 17 re-circulates gases to the high shear device 40. Recycling the unreacted gases from reactor 10 may serve to further accelerate the reactions.

Product stream 16 from the reactor 10 enters separator 30. Separator 30 comprises a filtration unit for separation of salts from product stream 16. Separator 30 removes traces of alkali metal salts previously introduced into reactor 10 in second inlet stream 15. The alkali salts are removed from separator 30 via wash stream 33, the remaining products comprise the oxidate stream 32. In embodiments wherein the second inlet stream 15 comprises ammonia, separation unit 30 may comprise a storage tank from which the aqueous compounds comprising wash stream 33 are separated from organic compounds comprising oxidate 32. Oxidate 32 may be further treated in order to separate the unreacted cumene from the cumene hydroperoxide and, if necessary, to concentrate the cumene peroxide until a content of a product stream of approximately 80 to 85% is obtained. Oxidate 32 is injected in to vaporizer 35 for distillation in least one distillation column 50. Unreacted cumene may be recovered from the distillation column 50 and the recovered cumene may be recycled through HSS 100 by recirculation stream 20. It may be necessary to treat the unreacted and recovered cumene 20 prior to recirculation, in order to remove any impurities, and particularly to remove of acid impurities.

CHP product stream 60 comprises a concentration of approximately 85% CHP. Concentrated CHP product stream 60 may be utilized as known to those of skill in the art. For example, in embodiments, CHP product stream 60 is decomposed to produce phenol and to acetone as known to those of skill in the art. The CHP contained in CHP product stream 60 may in be used, for example, in the reaction of CHP with alkanes to form detergent range alcohol and/or ketone in the presence of transition metal porphyrin catalyst as described in U.S. Pat. Nos. 4,978,799 and 4,970,346. Alternatively, conversion of CHP with alkanes to form detergent range alcohol and/or ketone in the presence of transition group metal catalyst is described in U.S. Patent Application No. 2006/0094905. Each of these patents is hereby incorporated herein in its entirety for all purposes.

In embodiments, not all the cumene introduced to the reactor 10 is converted to CHP. Generally, the degree of conversion of the cumene is between 20 wt % and 40 wt % such that cleavage of formed CHP is minimized. Condenser 70 on gas exit is configured for recovering unreacted cumene, whereby recovered cumene may be recycled through HSS 100 by recirculation stream 20. Alternatively, the unreacted cumene may be injected into waste gas stream 11 comprising oxygen for removal from HSS 100.

In embodiments, use of the disclosed process comprising reactant mixing via high shear device 40 allows faster production of CHP via oxidation of cumene. In embodiments, the method comprises incorporating high shear device 40 into an established process thereby enabling the increase in production, by greater throughput, compared to process operated without high shear device 40. The superior dissolution provided by the high shear mixing may allow a decrease in operating pressure while maintaining or even increasing reaction rate.

In embodiments, the method and system of this disclosure enable design of a smaller and/or less capital intensive process than previously possible without the incorporation of high shear device 40. In embodiments, the disclosed method reduces operating costs/increases production from an existing process. Alternatively, the disclosed method may reduce capital costs for the design of new processes.

The application of enhanced mixing of the reactants by high shear device 40 potentially causes greater conversion of cumene to cumene hydroperoxide in some embodiments of the process. Further, the enhanced mixing of the reactants potentiates an increase in throughput of the process stream of the high shear system 100. In certain instances, the high shear device 40 is incorporated into an established process, thereby enabling an increase in production (i.e., greater throughput).

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, and so forth). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of a reference in the Description of Related Art is not an admission that it is prior art to the present invention, especially any reference that may have a publication date after the priority date of this application. The disclosures of all patents, patent applications, and publications cited herein are hereby incorporated by reference, to the extent they provide exemplary, procedural or other details supplementary to those set forth herein.

We claim:

1. A method for producing cumene hydroperoxide, comprising
obtaining a high shear device having at least one rotor/stator set configured for producing a tip speed of at least 5 m/s;
forming an emulsion of cumene and air, wherein said air comprises gas bubbles with an average diameter less than about 5 μm;
and introducing said emulsion into a reactor wherein cumene hydroperoxide is produced.

2. The method of claim 1 wherein forming an emulsion comprises introducing air and cumene into a high shear device.

3. The method of claim 1 wherein said emulsion comprises gas bubbles having an average diameter of less than about 1.5 μm.

4. The method of claim 3 wherein the gas bubbles have a mean diameter of less than about 100 nm.

5. The method of claim 1 wherein the high shear device is configured to have a nominal tip speed of greater than about 23 m/s.

6. The method of claim 1 wherein said high shear device is configured to produce a localized pressure of at least about 1000 MPa at the tip.

7. The method of claim 1 including subjecting said cumene and gas bubbles to a shear rate of greater than about 20,000 s⁻¹.

8. The method of claim 1, wherein said high shear device is configured for an energy expenditure of at least 1000 W/m³.

9. The method of claim 1 including introducing an aqueous phase into the reactor with the emulsion.

10. The method of claim 9 wherein the aqueous phase comprises a byproduct neutralizing agent.

11. The method of claim 10 wherein the byproduct neutralizing agent is selected from a group comprising hydroxides and carbonates of alkali metals or alkaline earth metals.

12. The method of claim 10 wherein the neutralizing agent maintains the pH between about a pH 2 and about a pH 7.

13. The method of claim 10 wherein the aqueous phase comprises ammonia.

14. The method of claim 1 including maintaining the reactor at a temperature of at least about 75° C.

15. A system for the production of cumene hydroperoxide from air oxidation of cumene, comprising;
a pump positioned upstream of a high shear device, the pump in fluid connection with a high shear device inlet;
a high shear device which produces an emulsion of air in cumene, the emulsion having an average bubble diameter of less than about 1.5 μm; and
a reactor configured for the oxidation of cumene and production of cumene hydroperoxide at a temperature of at least about 75° C., the reactor fluidly connected to the outlet of the high shear device.

16. The system of claim 15 wherein the high shear device comprises a high shear mill having a nominal tip speed of greater than 5 m/s.

17. The system of claim 15 wherein the high shear mixer has a nominal tip speed of greater than 23 m/s.

18. The system of claim 15 wherein said high shear device is configured to produce a localized pressure of at least about 1000 MPa at the tip.

19. The system of claim 16 wherein said high shear device is configured to produce a shear rate of greater than about 20,000 s⁻¹.

20. The system of claim 16 wherein said high shear device is configured for an energy expenditure of at least 1000 W/m³.

21. The system of claim 16 wherein the reactor is configured to react a byproduct neutralizing agent.

22. The system of claim 21 wherein the byproduct neutralizing agent is selected from the group consisting of hydroxides and carbonates of alkali metals or alkaline earth metals.

23. The system of claim 16 wherein the reactor is configured to maintain the pH between about pH 2 and about pH 7.
